(19) United States
(12) Patent Application Publication
(10) Pub. No.: US 2014/0219931 A1
(43) Pub. Date: Aug. 7, 2014

(54) P-MENTHANE-3,8-DIOL ISOMER MIXTURE, COOLING SENSATION COMPOSITION COMPRISING THE SAME, AND PRODUCT COMPRISING THE COOLING SENSATION COMPOSITION

(71) Applicants: Yasuhiro Komatsuki, Kamakura-shi (JP); Shigeru Tanaka, Ota-ku (JP); Kenya Ishida, Yokohama-shi (JP)

(72) Inventors: Yasuhiro Komatsuki, Kamakura-shi (JP); Shigeru Tanaka, Ota-ku (JP); Kenya Ishida, Yokohama-shi (JP)

(73) Assignee: TAKASAGO INTERNATIONAL CORPORATION, Tokyo (JP)

(21) Appl. No.: 14/239,831

(22) PCT Filed: Aug. 31, 2012
(86) PCT No.: PCT/JP2012/072108

§ 371 Date: Feb. 20, 2014

(30) Foreign Application Priority Data
Aug. 31, 2011 (JP) 2011-189515

(51) Int. Cl. A61K 31/047; A61Q 11/00; A23G 4/06; A61Q 5/02; A23L 1/226; A61K 8/34; A61K 31/085

(52) U.S. Cl. CPC A61K 31/047; A61K 8/345; A61Q 11/00; A61K 31/085; A61Q 5/02; A23L 1/2265; A23G 4/06
USPC 424/49; 568/829; 514/729; 510/119; 512/5; 426/538; 426/3

(57) ABSTRACT

The present invention relates to a p-menthane-3,8-diol isomer mixture 50% by mass or more of which is constituted of (1S)-isomers and a cooling sensation composition containing this mixture. The present invention also relates to a flavor and/or fragrance composition, food, beverage, cosmetic, daily use product, oral cavity composition, or pharmaceutical containing the p-menthane-3,8-diol isomer mixture in an amount of 0.0001 to 90% by mass.

### TECHNICAL FIELD

The present invention relates to a p-menthane-3,8-diol isomer mixture and a cooling sensation composition comprising the isomer mixture. More specifically, the present invention relates to a p-menthane-3,8-diol isomer mixture 50% by mass or more of which is constituted of (1S)-isomers, and a cooling sensation composition comprising the isomer mixture.

Furthermore, the present invention relates to a sensory stimulant composition comprising the cooling sensation composition and a flavor and/or fragrance composition, food, beverage, cosmetic, daily use product, oral cavity composition, or pharmaceutical comprising the cooling sensation composition or the sensory stimulant composition.

### BACKGROUND ART

Conventionally, cooling sensation agents which provide a refreshing feeling (refreshing sensation) or a cool feeling (cooling sensation), namely a cooling sensation effect, to the skin, oral cavity, nose, or throat of a person has been used for tooth-cleaning agents, confectionery products (for example, chewing gums, candies, and the like), tobaccos, cataplasms, cosmetics, and the like. Currently, 1-menthol has been widely used as a substance which provides the refreshing sensation or the cooling sensation. However, 1-menthol has such drawbacks that the cooling sensation effect thereof is not persistent, and that the concentration of 1-menthol used and the application range thereof are limited because of its peculiar odor, bitterness, and the like.

In addition to 1-menthol, various compounds have been proposed and used as compounds having a cooling sensation effect. Examples of compounds, other than 1-menthol, having a cooling sensation effect and being proposed so far include 3-substituted-p-menthane (see, for example, Japanese Patent Application Publication No. Sho 47-16647), N-substituted-p-menthane-3-carboxamide (see, for example, Japanese Patent Application Publication No. Sho 47-16648), l-menthyl glucoside (see, for example, Japanese Patent Application Publication No. Sho 48-33069), 3-(1-menthoxy)propane-1,2-diol (see, for example, Japanese Patent Application Publication No. Sho 58-88334 and Japanese Patent Application Publication No. Sho 61-194049), 1-alkoxy-3-(1-menthoxy)propan-2-ol (see, for example, Japanese Patent Application Publication No. Hei 2-290827), esters of 3-hydroxymethyl-p-menthane (see, for example, Japanese Patent Application Publication No. Hei 5-255186), N-acetylglycine menthanemethyl ester (see, for example, Japanese Patent Application Publication No. Hei 5-255217), (−)-isopulegol (see, for example, Japanese Patent Application Publication No. Hei 6-65023), (2S)-3-(1-menthoxy)propane-1,2-diol (see, for example, Japanese Patent Application Publication No. Hei 7-82200), 2-hydroxymethylmenthol (see, for example, Japanese Patent Application Publication No. Hei 7-118119), and the like. However, there is a demand for development of a cooling sensation agent which satisfies all requirements such as the strength and persistence of the cooling sensation and further an excellent flavor with less foreign tastes such as bitterness.

### SUMMARY OF INVENTION

Accordingly, an object of the present invention is to provide a p-menthane-3,8-diol isomer mixture as a cooling sensation component which is free from unfavorable stimulation, especially bitterness, and which is excellent in the cooling sensation strength and in the cooling sensation persistence, and a cooling sensation composition comprising the p-menthane-3,8-diol isomer mixture.

Moreover, another object of the present invention is to provide a sensory stimulant composition comprising the cooling sensation composition, and a flavor and/or fragrance composition, food, beverage, cosmetic, daily use product, oral cavity composition, or pharmaceutical comprising the cooling sensation composition or the sensory stimulant composition.

The present inventors have conducted earnest study to solve the above-described problems, and found that a p-menthane-3,8-diol isomer mixture which is represented by the following formula (I) and 50% by mass or more of which is constituted of (1S)-isomers is excellent in the cooling sensation strength or the persistence of the cooling sensation effect, and is useful as a cooling sensation component and further as a sensory stimulation substance. The present invention has been completed on the basis of these findings.

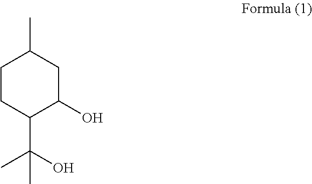

Specifically, the present invention relates to a cooling sensation composition comprising a p-menthane-3,8-diol isomer mixture 50% by mass or more of which is constituted of (1S)-isomers.

Moreover, the present invention relates to a sensory stimulant composition comprising: the cooling sensation composition; and at least one warming sensation component selected from the group consisting of capsaicin, gingerol, vanillyl butyl ether, vanillyl ethyl ether, vanillyl propyl ether, 4-(1-menthoxymethyl)-2-phenyl-1,3-dioxolane, 4-(1-menthoxymethyl)-2-(3′,4′-dihydroxyphenyl)-1,3-dioxolan e, 4-(1-menthoxymethyl)-2-(2′-hydroxy-3′-methoxyphenyl)-1,3-di oxolane, 4-(1-menthoxymethyl)-2-(4′-methoxyphenyl)-1,3-dioxolane, 4-(1-menthoxymethyl)-2-(3′,4′-methylenedioxyphenyl)-1,3-dioxolane, 4-(1-methoxymethyl)-2-(3′-methoxy-4′-hydroxyphenyl)-1,3-dio xolane, vanillin acetals, capsicum pepper oil, capsicum pepper oleoresin, ginger oleoresin, nonylic acid vanillyl amide, jambu oleoresin, Japanese pepper extract, sanshool-I, sanshool-II, sanshoamide, black pepper extract, chavicine, piperine, and spilanthol.

Further, the present invention relates to a flavor and/or fragrance composition, food, beverage, cosmetic, daily use product, oral cavity composition, or pharmaceutical comprising the cooling sensation composition or the sensory stimulant composition.

Moreover, the present invention relates to a p-menthane-3,8-diol isomer mixture 50% by mass or more of which is constituted of (1S)-isomers.

The present invention also relates to a flavor and/or fragrance composition, food, beverage, cosmetic, daily use product, oral cavity composition, or pharmaceutical comprising the p-menthane-3,8-diol isomer mixture in an amount of 0.0001 to 90% by mass.

The p-menthane-3,8-diol represented by the above-described formula (I) does not have peculiar odor or the like. In addition, when blended with various foods, beverages, oral cavity compositions, cosmetics, daily use products, pharmaceuticals, and the like, the p-menthane-3,8-diol can make these products capable of providing a refreshing sensation or a cooling sensation, and can make these products capable of providing a persistent and excellent refreshing sensation or cooling sensation. Moreover, the p-menthane-3,8-diol exhibits an excellent characteristic of causing substantially no skin stimulation, which is unfavorable for the human body, and is not colored during storage and has excellent stability.

In addition, the p-menthane-3,8-diol isomer mixture 50% by mass or more of which is constituted of (1S)-isomers exhibits a cooling sensation 2 to 5 times as strong as and 1.5 to 3 times as persistent as that of (1R)-p-menthane-3,8-diol, which is widely used in general, while retaining a characteristic of being less bitter than (1R)-p-menthane-3,8-diol. When this p-menthane-3,8-diol isomer mixture is used for a flavor or the like, for example, when this p-menthane-3,8-diol isomer mixture is used for a chewing gum, a candy, or the like, an oral cavity care product such as a tooth-cleaning agent or a mouth wash, it is no more necessary to avoid the use thereof or to limit the amount thereof used.

### DESCRIPTION OF EMBODIMENTS

Hereinafter, embodiments of the present invention are described in detail.

### p-Menthane-3,8-diol Isomer Mixture

Several methods have been reported so far for synthesizing p-menthane-3,8-diol represented by the above-described formula (I). For example, Japanese Patent Application Publication No. 2000-44924 reports a method for producing para-menthane-3,8-diol, the method comprising treating citronellal with an aqueous sulfuric acid solution of a concentration of 0.02 to 1.0% by mass.

It has been a common practice so far that p-menthane-3,8-diol is produced from d-citronellal, which is placed on the market in large quantities. However, the cooling sensation effect and the cooling sensation persistence of (1R)-p-menthane-3,8-diol, which is produced from d-citronellal, are not sufficiently satisfactory, when it is used alone.

In addition, p-menthane-3,8-diol has three asymmetric carbons, and hence has 8 stereoisomers. There has been no report so far on a case where the difference in cooling sensation among these isomers is examined.

Now, the present inventors have found, as described above, that a p-menthane-3,8-diol isomer mixture 50% by mass or more of which is constituted of (1S)-isomers is excellent in cooling sensation strength or persistence of the cooling sensation effect. This p-menthane-3,8-diol isomer mixture exhibits a cooling sensation 2 to 5 times as strong as and 1.5 to 3 times as persistent as that of the (1R)-p-menthane-3,8-diol, which is widely used in general, while retaining such a characteristic of being less bitter than (1R)-p-menthane-3,8-diol. Here, the (1S)-isomers constitute preferably 60% by mass or more, more preferably 70% by mass or more, further preferably 80% by mass or more, and particularly preferably 90% by mass or more of the p-menthane-3,8-diol isomer mixture.

Moreover, in the p-menthane-3,8-diol isomer mixture, the mass ratio of the (1S,3R,4S) isomer:the (1R,3S,4R) isomer is preferably 50:50 to 99.5:0.5. This is because of the following reasons. Specifically, if the mass ratio value of the (1S,3R,4S) isomer in the mass ratio of the (1S,3R,4S) isomer:the (1R,3S,4R) isomer is less than 50, there is a possibility that the above-described effects of the high cooling sensation strength and the low bitterness cannot be achieved. If the mass ratio of the (1S,3R,4S) isomer exceeds 99.5, there is a possibility that a process for increasing the optical purity is required, so that the process is complicated. In addition, in the p-menthane-3,8-diol isomer mixture, the mass ratio of the (1S,3R,4S) isomer:the (1R,3S,4R) isomer is more preferably 60:40 to 99.5:0.5, further preferably 70:30 to 99.5:0.5, still further preferably 80:20 to 99.5:0.5, and particularly preferably 90:10 to 99.5:0.5.

Furthermore, the (1S,3R,4S) isomer and the (1S,3S,4S) isomer constitute preferably 90% by mass or more and more preferably 95% by mass or more of the entire composition of the p-menthane-3,8-diol isomer mixture of the present invention. This is because of the following reasons. Specifically, it is relatively easy to produce a p-menthane-3,8-diol isomer mixture in which, among the four (1S)-isomers of p-menthane-3,8-diol, the (1S,3R,4S) isomer and the (1S,3S,4S) isomer constitute the majority. Hence, the process can be simplified. Moreover, the p-menthane-3,8-diol isomer mixture, wherein the (1S,3R,4S) isomer and the (1S,3S,4S) isomer constitute 90% by mass or more of the entire composition, has a very high cooling sensation strength, but an extremely low bitterness.

Note that a method for producing (1S)-isomers of p-menthane-3,8-diol is not particularly limited. For example, it is possible to employ a method known to those skilled in the art in which d-citronellal is used as a raw material. In this case, a p-menthane-3,8-diol isomer mixture can be obtained in which the (1S,3R,4S) isomer constitutes 60 to 70% by mass of the entire composition, and the (1S,3S,4S) isomer constitutes 30 to 40% by mass of the entire composition. The p-menthane-3,8-diol isomer mixture having such a composition can be obtained efficiently, and has a very high cooling sensation strength, a very high cooling sensation persistence, and a very low bitterness.

In addition, in the present invention, when the p-menthane-3,8-diol isomer mixture 50% by mass or more of which is constituted of (1S)-isomers alone or in the form of a cooling sensation composition or a sensory stimulant composition as described later is contained in a product, the p-menthane-3,8-diol isomer mixture is preferably used at a concentration of 0.0001 to 90% by mass and particularly 0.01 to 20% by mass relative to the entire composition of the product, in general. However, it is necessary to change, as appropriate, the range and method of application, depending on the kind, purpose of use, and the like of the product.

### <Cooling Sensation Composition>

The present invention also relates to a cooling sensation composition comprising the above-described p-menthane-3,8-diol isomer mixture. In addition to the p-menthane-3,8-diol isomer mixture, various components can be mixed with the cooling sensation composition depending on the purpose of use.

Moreover, from the p-menthane-3,8-diol isomer mixture of the present invention 50% by mass or more of which is constituted of (1S)-isomers, a cooling sensation composition having an enhanced cooling sensation strength or a sensory stimulant composition described later can be prepared by using at least one selected from cooling sensation components other than p-menthane-3,8-diol in combination.

Examples of the cooling sensation components other than the p-menthane-3,8-diol isomer mixture of the present invention include menthol, isopulegol, menthone, camphor, pulegol, cineol, mint oil, 3-menthoxypropane-1,2-diol, N-alkyl-p-menthane-3-carboxamide, 3-menthoxy-2-methylpropane-1,2-diol, 2-(menthoxy)ethanol, 2-methyl-3-(1-menthoxy)propane-1,2-diol, 3-menthoxypropan-1-ol, 4-1-menthoxybutan-1-ol, menthyl 3-hydroxybutanoate, 1-(2-hydroxy-4-methylcyclohexyl)-ethanone, menthyl lactate, menthol glycerin ketal, N-methyl-2,2-isopropylmethyl-3-methylbutanamide, menthyl glyoxylate, menthyl succinate, menthyl glutarate, peppermint oil, spearmint oil, eucalyptus oil, mint oil, peppermint, spearmint, and the like. One of these cooling sensation components or a suitable blend of two or more thereof can be used. Among these cooling sensation components, it is particularly preferable to use menthol, isopulegol, 3-menthoxypropane-1,2-diol, 2-(menthoxy)ethanol, or menthyl 3-hydroxybutanoate, because a desired cooling sensation effect can be provided freely.

In the cooling sensation composition of the present invention, the p-menthane-3,8-diol isomer mixture and the cooling sensation components other than p-menthane-3,8-diol can be used at any ratio, as long as the effects of the present invention are not impaired. The mass ratio of p-menthane-3,8-diol to the other cooling sensation components is preferably in a range from 1:99 to 95:5. The cooling sensation composition of the present invention can be blended with a flavor and/or fragrance composition, food, beverage, cosmetic, daily use product, oral cavity composition, or pharmaceutical.

### <Sensory Stimulant Composition>

The present invention also relates to a sensory stimulant composition comprising the above-described cooling sensation composition.

Here, the sensory stimulant composition of the present invention means a composition which provides an effect of stimulating a sense. The effect of stimulating a sense includes a cooling sensation effect and a warming sensation effect. Hence, in the present invention, the sensory stimulant composition can be used as a concept encompassing the above-described cooling sensation composition and a warming sensation composition which provides a warming sensation effect to the skin, oral cavity, nose, throat of a person.

Since the p-menthane-3,8-diol isomer mixture of the present invention has a strong and persistent cooling sensation effect, a sensory stimulant composition having a cooling sensation effect can be prepared by adding the cooling sensation composition comprising the p-menthane-3,8-diol isomer mixture. When the sensory stimulant composition is prepared, the amount of the p-menthane-3,8-diol isomer mixture blended is preferably such that the p-menthane-3,8-diol isomer mixture is used at a concentration of 0.0001 to 20% by mass and particularly 0.001 to 5% by mass relative to the entire composition of the sensory stimulant composition, in general. However, it is necessary to change, as appropriate, the range and method of application, depending on the application of the sensory stimulant composition, for example, the kind, purpose of use, and the like of the product.

A sensory stimulant composition can be prepared and a stimulation effect of the sensory stimulant composition can be adjusted by using the cooling sensation composition of the present invention comprising the p-menthane-3,8-diol isomer mixture in combination with a warming sensation component. The warming sensation component include vanillyl ethyl ether, vanillyl propyl ether, vanillin propylene glycol acetal, ethyl vanillin propylene glycol acetal, capsaicin, gingerol, vanillyl butyl ether, 4-(1-menthoxymethyl)-2-phenyl-1,3-dioxolane, 4-(1-menthoxymethyl)-2-(3′,4′-dihydroxyphenyl)-1,3-dioxolane, 4-(1-menthoxymethyl)-2-(2′-hydroxy-3′-methoxyphenyl)-1,3-dioxolane, 4-(1-menthoxymethyl)-2-(4′-methoxyphenyl)-1,3-dioxolane, 4-(1-menthoxymethyl)-2-(3′,4′-methylenedioxyphenyl)-1,3-dioxolane, 4-(1-methoxymethyl)-2-(3′-methoxy-4′-hydroxyphenyl)-1,3-dioxolane, vanillin acetals, capsicum pepper oil, capsicum pepper oleoresin, ginger oleoresin, nonylic acid vanillyl amide, jambu oleoresin, Japanese pepper extract, sanshool-I, sanshool-II, sanshoamide, black pepper extract, chavicine, piperine, spilanthol, and the like. One of these warming sensation components or a suitable blend of two or more thereof can be used. Among these warming sensation components, it is particularly preferable to use vanillyl butyl ether, vanillyl ethyl ether, or spilanthol, because a desired sensory stimulation can be provided freely.

When the sensory stimulant composition is intended for a cooling sensation effect, the content of the warming sensation component in the sensory stimulant composition only needs to be within a range where the warming sensation component does not provide the warming sensation effect, and the mass of the warming sensation component is generally set to be 0.001 to 0.95 times and preferably 0.003 to 0.5 times the total mass of the p-menthane-3,8-diol isomer mixture and the cooling sensation component. In this case, the addition of the warming sensation component to the cooling sensation composition in the sensory stimulant composition of the present invention at the above-described ratio further improves the cooling sensation effect and increases the cooling sensation effect.

Meanwhile, when the sensory stimulant composition is intended for a warming sensation effect, the content of the p-menthane-3,8-diol isomer mixture and the cooling sensation component in the sensory stimulant composition only needs to be within a range where these components do not provide a cooling sensation effect, and the mass of these components is generally set to be 0.001 to 0.95 times and preferably 0.01 to 0.5 times the total mass of the warming sensation component.

### <Products>

The present invention also relates to products comprising the above-described cooling sensation composition or the above-described sensory stimulant composition comprising the p-menthane-3,8-diol isomer mixture. In the present invention, the cooling sensation composition or the sensory stimulant composition may be blended directly with various products such as flavor and/or fragrance compositions, foods, beverages, cosmetics, daily use products, oral cavity compositions, and pharmaceuticals. Alternatively, the cooling sensation composition or the sensory stimulant composition is first blended with a flavor and/or fragrance or a flavor and/or fragrance composition to form a cooling sensation composition-containing or sensory stimulant composition-containing flavor and/or fragrance composition (hereinafter also referred to simply as a “flavor and/or fragrance composition according to the present invention” or a “flavor and/or fragrance composition”), and the flavor and/or fragrance composition according to the present invention can be blended with products. Here, it is also possible to add p-menthane-3,8-diol, the other cooling sensation component, and the warming sensation component to separate flavor and/or fragrance compositions, respectively, and mix these flavor and/or fragrance compositions with a product.

As described above, the cooling sensation composition or the sensory stimulant composition of the present invention can be used alone or in the form of the flavor and/or fragrance composition according to the present invention for making various products capable of providing a cooling sensation or a sensory stimulation. Examples of products which can be made capable of providing a cooling sensation or a sensory stimulation by the cooling sensation composition or the sensory stimulant composition of the present invention itself or the flavor and/or fragrance composition according to the present invention include foods, beverages, cosmetics, daily use products, oral cavity compositions, pharmaceuticals, and the like.

<<Flavor and/or Fragrance Composition>>

Flavor and/or fragrance components which can be contained in the flavor and/or fragrance composition according to the present invention in combination with the cooling sensation composition or the sensory stimulant composition include various synthetic flavors and fragrances, natural essential oils, synthetic essential oils, citrus oils, animal flavors and fragrances, and the like. For example, a wide variety of flavor and/or fragrance components such as those described in “Collection of Well-known Prior Arts (flavors and fragrances), Part I” (issued by Japan Patent Office on Jan. 29, 1999) can be used.

Of these components, typical examples are α-pinene, limonene, neral, geranial, 2,5-dimethyl-4-hydroxy-3(2H)-furanone, vanillin, ethylvanillin, geraniol, nerol, citronellol, cis-3-hexenol, phenylethyl alcohol, styralyl acetate, eugenol, rose oxide, linalool, benzaldehyde, muscone, Musk T (Takasago International Corporation), Thesaron (Takasago International Corporation), and the like.

The content of the cooling sensation composition or the sensory stimulant composition in the flavor and/or fragrance composition according to the present invention can be adjusted depending on the kinds of flavors and/or fragrances and other components blended in combination, the purpose of use of the flavor and/or fragrance composition according to the present invention, and the like. For example, in a flavor and/or fragrance composition for a cosmetic, the content of the cooling sensation composition or the sensory stimulant composition is preferably 0.0001 to 90% by mass, more preferably 0.001 to 70% by mass, and particularly preferably 0.01 to 50% by mass relative to the total mass of the flavor and/or fragrance composition, in general.

In other words, the flavor and/or fragrance composition contains the p-menthane-3,8-diol isomer mixture in an amount of preferably 0.0001 to 90% by mass and further preferably 0.01 to 50% by mass.

Meanwhile, in a flavor and/or fragrance composition for a food or beverage, the content of the cooling sensation composition or the sensory stimulant composition is preferably 0.0001 to 90% by mass and more preferably 0.01 to 50% by mass relative to the total mass of the flavor and/or fragrance composition, in general. Further, the same content is preferable in the case of a flavor and/or fragrance composition for a daily use product, an oral cavity composition, or a pharmaceutical.

In other words, these flavor and/or fragrance compositions contain the p-menthane-3,8-dial isomer mixture in an amount of preferably 0.0001 to 90% by mass and further preferably 0.01 to 50% by mass.

If necessary, the flavor and/or fragrance composition according to the present invention may contain one or more flavor- and/or fragrance-retaining agents generally used for ordinary flavor and/or fragrance compositions. In this case, examples of the flavor- and/or fragrance-retaining agents include ethylene glycol, propylene glycol, dipropylene glycol, glycerin, hexylglycol, benzyl benzoate, triethyl citrate, diethyl phthalate, HERCOLYN, medium-chain fatty acid triglycerides, medium-chain fatty acid diglycerides, and the like. The flavor and/or fragrance composition according to the present invention can contain one or more of these flavor- and/or fragrance-retaining agents.

### <<Food and Beverage>>

Specific examples of foods and beverages which can be made capable of providing a cooling sensation or a sensory stimulation by the cooling sensation composition or the sensory stimulant composition of the present invention or the flavor and/or fragrance composition comprising the cooling sensation composition or the sensory stimulant composition include beverages such as fruit juice beverages, alcoholic fruit beverages, dairy beverages, carbonated beverages, refreshing beverages, and drink preparations; cold desserts such as ice creams, sherbets, and ice pops; desserts such as jellies and créme caramels; Western confectionery products such as cakes, cookies, chocolates, and chewing gums; Japanese confectionery products such as steamed bean-jam buns, Yokan (a thick jellied dessert made of red bean paste, agar, and sugar), and Uiro (a traditional Japanese steamed cake made of rice flour and sugar); jams; candies; breads; tea beverages and other favorite beverages such as green tea, oolong tea, black tea, persimmon leaf tea, chamomile tea, Sasa veitchii tea, mulberry leaf tea, Houttuynia cordata tea, pu-erh tea, mate tea, rooibos tea, gymnema tea, guava tea, coffee, and cocoa; soups such as Japanese style soups, Western style soups, and Chinese style soups; flavor seasonings; foods such as various instant beverages; various snack foods; and the like. However, the foods and beverages are not limited to these examples.

### <<Cosmetic and Daily Use Product>>

Examples of cosmetics and daily use products which can be scented with the cooling sensation composition or the sensory stimulant composition of the present invention or the flavor and/or fragrance composition comprising the cooling sensation composition or the sensory stimulant composition include fragrance products, basic skin care cosmetics, make-up cosmetics, hair cosmetics, sunscreen cosmetics, medicated cosmetics, hair-care products, soaps, body cleaning agents, bath agents, detergents, finishing softeners, cleaning agents, kitchen detergents, bleaching agents, aerosol agents, deodorant and/or aromatic agents, repellents, other utensil products, and the like.

More specifically,

the fragrance products include perfumes, Eau de Parfum, Eau de Toilette, Eau de Cologne, and the like;

the basic skin care cosmetics include face wash creams, vanishing creams, cleansing creams, cold creams, massage creams, emulsions, lotions, cosmetic serums, beauty packs, make-up removers, and the like;

the make-up cosmetics include foundations, loose face powders, pressed face powders, talcum powders, lipsticks, lip balms, cheek rouges, eyeliners, mascaras, eye shadows, eyebrow colors, eye packs, nail enamels, enamel removers, and the like; and

the hair cosmetics include pomades, brilliantines, set lotions, hair sticks, hair solids, hair oils, hair treatments, hair creams, hair tonics, hair liquids, hair sprays, bandolines, hair-growing agents, hair dyes, and the like.

The sunscreen cosmetics include suntan products, sunscreen products, and the like;

the medicated cosmetics include antiperspirant, after-shaving lotions and gels, permanent wave agents, medicated soaps, medicated shampoos, medicated skin cosmetics, and the like;

the hair-care products include shampoos, rinses, two-in-one shampoos, conditioners, treatments, hair packs, and the like;

the soaps include toilet soaps, bath soaps, perfume soaps, transparent soaps, synthetic soaps, and the like;

the body cleaning agents include body soaps, body shampoos, hand soaps, and the like;

the bath agents include bath additives (bath salts, bath tablets, bath liquids, and the like), foam baths (bubble baths and the like), bath oils (bath perfumes, bath capsules, and the like), milk baths, bath jellies, bath cubes, and the like; and

the detergents include heavy or light duty detergents, liquid detergents, laundry soaps, compact detergents, powder soaps, and the like.

The finishing softeners include softeners, furniture cares, and the like;

the cleaning agents include cleansers, house cleaning agents, toilet cleaners, bath cleaners, glass cleaners, mold removers, drain pipe cleaners, and the like;

the kitchen detergents include kitchen soaps, kitchen synthetic soaps, dish detergents, and the like;

the bleaching agents include oxidation bleaching agents (chlorine-based bleaching agents, oxygen-based bleaching agents, and the like), reduction bleaching agents (sulfur-based bleaching agents, and the like), optical bleaching agents, and the like;

the aerosol agents include spray-type aerosol agents, powder sprays, and the like;

the deodorant and/or aromatic agents include solid-, gel- or liquid-type deodorant and/or aromatic agents, and the like; and

the repellents include emulsions, oil solutions, wettable powders, sprays, liniments, powders, pellets, capsules, sheets, and the like.

A repellent comprising the cooling sensation composition or the sensory stimulant composition comprising the p-menthane-3,8-diol isomer mixture may further comprise any of N,N-diethyl-m-toluamide, 1-methylpropyl 2-(2-hydroxyethyl)-1-piperidinecarboxylate, 2,3,4,5-bis(A2-butylene)tetrahydrofurfural, 3,4-caranediol, di-n-propyl isocinchoronate, 3-(N-n-butyl-N-acetyl)aminopropionic acid ethyl ester di-n-butyl succinate, 2-ethyl-1,3-hexanediol, 2-hydroxyoctyl sulfide, dimethyl phthalate, and (N-carbo-sec-butyloxy)-2-(2′-hydroxyethyl) piperidine, as well as any of plant essential oils such as citronella oil, lemongrass oil, hyssop oil, horseradish oil, bay oil, Angelica dahurica oil, fennel oil, basil oil, star anise oil, calamus oil, oregano oil, sandalwood oil, yarrow oil, perilla oil, valerian oil, ginger oil, palmarosa oil, laurel oil, ylang-ylang oil, and clove oil, or the like.

The utensil products include tissue paper, toilet paper, and the like.

### <<Oral Cavity Composition and Pharmaceutical>>

Examples of the oral cavity compositions include tooth-cleaning agents (toothpastes and tooth gels), oral cavity washes, mouthwashes, troches, chewing gums, and the like.

The pharmaceuticals include skin external agents such as cataplasms and ointments, internal preparations, and the like.

### <<Form of Composition Used for Product>>

When the cooling sensation composition or the sensory stimulant composition of the present invention or the flavor and/or fragrance composition comprising the cooling sensation composition or the sensory stimulant composition is used for making various products capable of providing a cooling sensation or a sensory stimulation as described above, the cooling sensation composition or the sensory stimulant composition or the flavor and/or fragrance composition comprising the cooling sensation composition or the sensory stimulant composition may be added in various manners depending on the kind of the product which is made capable of providing a cooling sensation or a sensory stimulation, and the final form of the product (for example, the form of the product such as liquid, solid, powder, gel, mist, or aerosol). Specifically, these may be added directly to the product. Alternatively, the cooling sensation composition, the sensory stimulant composition, or the cooling sensation-containing or sensory stimulant composition-containing flavor and/or fragrance composition may be added in a liquid from, for example, after being dissolved in a monohydroxy alcohol or a polyol such as propylene glycol or glycerin. Further, they may be added in a soluble form or a dispersed form, after being converted into a soluble form or being dispersed by emulsification by using a naturally occurring gum substance such as gum arabic or gum tragacanth or a surfactant (for example, a nonionic surfactant such as a glycerin fatty acid ester or a sucrose fatty acid ester, an anionic surfactant, a cationic surfactant, an amphoteric surfactant, or the like). Moreover, they may be added in a powder form, after coating is formed by using an excipient such as a naturally occurring gum substance such as gum arabic, gelatin, dextrin, or the like. Further, they may be added in a form of microcapsules after being treated with a capsule-forming agent. Moreover, the cooling sensation composition, the sensory stimulant composition, or the flavor and/or fragrance composition comprising the cooling sensation composition or the sensory stimulant composition may be used in a stabilized and sustained-release form, after being included in an inclusion agent such as cyclodextrin.

The amount of the cooling sensation composition or the sensory stimulant composition added to the product for making the product capable of providing a cooling sensation or a sensory stimulation can be adjusted depending on the kind and form of the product, effects and operations of providing a cooling sensation or a sensory stimulation required by the product, and the like. In general, the amount of the cooling sensation composition or the sensory stimulant composition added is preferably 0.0001 to 90% by mass and, in particular, more preferably 0.01 to 20% by mass, relative to the mass of the product.

In other words, the product contains the p-menthane-3,8-diol isomer mixture in an amount of preferably 0.0001 to 90% by mass and further preferably 0.01 to 20% by mass.

### Examples

Hereinafter, the present invention is described more specifically based on examples. However, the present invention is not limited to these examples at all. Any modifications may be made as appropriate within the scope of the present invention. Note that the following apparatus was used for identification and physical property measurement of products of examples and test experiments.

GC: GC-4000 of GL Sciences Inc.

### Example 1

### Synthesis of (1S)-p-Menthane-3,8-diol Isomer Mixture

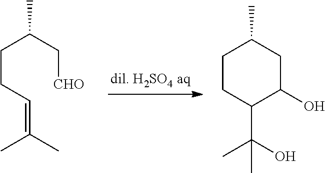

Into a 4-necked 500-ml flask equipped with a stirrer, a thermometer, and a dropping tube, 133.7 g of an aqueous sulfuric acid solution of a concentration of 0.25% by mass and 93 ml of toluene were introduced, and heated to 70° C. To this mixture, 77.1 g of 1-citronellal (manufactured by Takasago International Corporation) was added dropwise over 1 hour, and then the reaction was allowed to proceed at the same temperature for 10 hours. The conversion of citronellal was 98.2%. The reaction liquid was neutralized by adding a 25% aqueous sodium hydroxide solution, and subjected to an extraction treatment by adding 150 ml of n-heptane followed by stirring.

The organic layer was recovered by separation from the aqueous layer, and washed with water, followed by phase separation. Further, the solvent was removed by distillation from the organic layer, followed by vacuum distillation. Thus, 67.5 g of a (1S)-p-menthane-3,8-diol isomer mixture was obtained. In this isomer mixture, (1S)-isomers accounted for 99% by mass of the entire composition (more specifically, the (1S,3R,4S) isomer accounted for 66% by mass of the entire composition, and the (1S,3S,4S) isomer accounted for 30% by mass of the entire composition).

### Example 2

### Synthesis of (IRS)-p-Menthane-3,8-diol Isomer Mixture

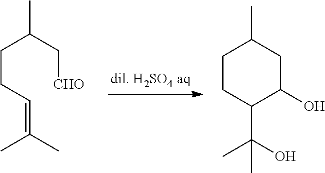

By using 77.1 g of dl-citronellal (manufactured by Takasago International Corporation) containing 50% by mass of d-citronellal and 50% by mass of 1-citronellal as a raw material, 64.2 g of a (1RS)-p-menthane-3,8-diol isomer mixture was obtained by the same synthetic method as in [Example 1]. In this isomer mixture, (1R)-isomers accounted for 50% by mass of the entire composition, and (1S)-isomers accounted for 50% by mass of the entire composition (more specifically, the (1S,3R,4S) isomer accounted for 33% by mass of the entire composition, and the (1R,3S,4R) isomer accounted for 33% by mass of the entire composition).

### Comparative Example 1

### Synthesis of (1R)-p-Menthane-3,8-diol Isomer Mixture

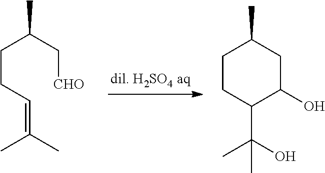

By using 77.1 g of d-citronellal (manufactured by Takasago International Corporation) as a raw material, 65.9 g of a (1R)-p-menthane-3,8-diol isomer mixture was obtained by the same synthetic method as in [Example 1]. In this isomer mixture, (1R)-isomers accounted for 99% by mass of the entire composition (specifically, the (1R,3S,4R) isomer accounted for 66% by mass of the entire composition, and the (1R,3R,4R) isomer accounted for 30% by mass of the entire composition).

[Test Experiment 1] Sensory Evaluation for Comparison of Cooling Sensation Strength between (1S)-p-Menthane-3,8-diol Isomer Mixture, (1RS)-p-Menthane-3,8-diol Isomer Mixture, and (1R)-p-Menthane-3,8-diol Isomer Mixture

Aqueous solutions containing 180 ppm of the (1S)-p-menthane-3,8-diol isomer mixture, the (1RS)-p-menthane-3,8-diol isomer mixture, and the (1R)-p-menthane-3,8-diol isomer mixture obtained in Example 1, Example 2, and Comparative Example 1, respectively, were prepared. Sensory evaluation was conducted in the mouth on these 180 ppm aqueous solutions by 10 professional panelists having 5-year or longer experience.

As a result, all the 10 panelists stated that the (1S)-p-menthane-3,8-diol isomer mixture provided the strongest refreshing sensation, and the refreshing sensation was 4 to 5 times as strong as that of the (1R)-p-menthane-3,8-diol isomer mixture. In addition, all the panelists stated that the (1RS)-p-menthane-3,8-diol isomer mixture provided a cooling sensation 2 to 3 times as strong as that of the (1R)-p-menthane-3,8-diol isomer mixture.

In other words, the relative average cooling sensation strengths of the p-menthane-3,8-diol isomer mixtures are as shown in Table 1, where the cooling sensation strength of the isomer mixture of Comparative Example 1 is taken as 3.

TABLE 1

| | Isomer | Isomer | Isomer |
|---|---|---|---|
| | mixture of | mixture of | mixture of |
| | Comp. Ex. 1 | Ex. 1 | Ex. 2 |
| Average cooling | 3 | 15 | 9 |
| sensation strength | | | |
| score | | | |

[Test Experiment 2] Sensory Evaluation for Comparison of Cooling Sensation Persistence between (1S)-p-Menthane-3,8-diol Isomer Mixture, (1RS)-p-Menthane-3,8-diol Isomer Mixture, and (1R)-p-Menthane-3,8-diol Isomer Mixture

Aqueous solutions containing 180 ppm of the (1S)-p-menthane-3,8-diol isomer mixture, the (1RS)-p-menthane-3,8-diol isomer mixture, and the (1R)-p-menthane-3,8-diol isomer mixture obtained in Example 1, Example 2, and Comparative Example 1, respectively, were prepared. Sensory evaluation was conducted in the mouth on these 180 ppm aqueous solutions by 10 professional panelists having 5-year or longer experience.

In the sensory evaluation, each 180 ppm aqueous solution was retained in the month for 30 seconds, and then spit out. After that, the time taken until the cooling sensation disappeared was measured, and the average was calculated to evaluate the effect. Table 2 shows the result.

TABLE 2

| | Isomer | Isomer | Isomer |
|---|---|---|---|
| | mixture of Ex. | mixture of | mixture of |
| | 1 | Ex. 2 | Comp. Ex. 1 |
| Cooling sensation | 15.5 | 9.8 | 4.3 |
| persistence time | | | |
| (minute) | | | |

This result showed that the (1S)-p-menthane-3,8-diol isomer mixture had a cooling sensation effect approximately 3.6 times as persistent as that of the (1R)-p-menthane-3,8-diol isomer mixture, and the (1RS)-p-menthane-3,8-diol isomer mixture had a cooling sensation effect approximately 2.3 times as persistent as that of the (1R)-p-menthane-3,8-diol isomer mixture.

### Example 3

### Cooling Sensation Composition (Test for Synergistic Effect with Menthol)

A cooling sensation composition was prepared by mixing l-menthol and the (1S)-p-menthane-3,8-diol isomer mixture obtained in Example 1 at 95:5 (mass ratio). An aqueous solution containing 20 ppm of the obtained cooling sensation composition was prepared, and sensory evaluation was conducted in the mouth on the aqueous solution. In addition, for comparison, sensory evaluation was conducted in the mouth also on an aqueous solution containing 20 ppm of 1-menthol alone.

Note that the evaluations were conducted by 10 professional panelists having 5-year or longer experience. Specifically, 10 ml of each aqueous solution was retained in the mouth for 10 seconds, and then spit out. The time point of the spitting out was taken as 0 seconds, and the strength of the refreshing property (refreshing feel) was evaluated at this time point. In addition, the persistence of the strength of the refreshing property (refreshing feel) was evaluated 3 minutes later.

As a result, 9 out of the 10 panelists stated that, immediately after the spitting out, the solution containing the (1S)-p-menthane-3,8-diol isomer mixture exhibited a stronger refreshing property (refreshing feel) and a weaker bitterness than the solution containing menthol alone.

In addition, 8 out of the 10 panelists stated that, after 3 minutes had elapsed from the spitting out, the solution containing the (1S)-p-menthane-3,8-diol isomer mixture was more persistent in the strong refreshing property than the solution containing menthol alone.

### Example 4

### Sensory Stimulant Composition (Test for Synergistic Effect of Addition of Vanillyl Butyl Ether)

A sensory stimulant composition was prepared which was the (1S)-p-menthane-3,8-diol isomer mixture obtained in Example 1 and containing 0.5% by mass of a warming sensation component vanillyl butyl ether.

1,000 ml of an aqueous solution containing 20 ppm of the prepared sensory stimulant composition was prepared, and sensory evaluation was conducted in the mouth on the aqueous solution. For comparison, sensory evaluation was conducted in the mouth on an aqueous solution containing 20 ppm of the (1S)-p-menthane-3,8-dial isomer mixture alone.

Note that the evaluation was conducted by 10 professional panelist having 5-year or longer experience. Specifically, 10 ml of the aqueous solution was retained in the mouth for 10 seconds, and then spit out. The time point of the spitting out was taken as 0 seconds, and the strength of refreshing property (refreshing feel) was evaluated at the time point. In addition, the persistence of the strength of the refreshing property (refreshing feel) was evaluated 3 minutes later.

As a result, all the 10 panelists stated that, at 0 seconds (immediately after the spitting out), the solution containing vanillyl butyl ether exhibited a stronger refreshing property (refreshing feel) than the solution containing the (1S)-p-menthane-3,8-diol isomer mixture alone. Moreover, all the 10 panelists stated that, also 3 minutes later, the solution containing vanillyl butyl ether exhibited a stronger refreshing property (refreshing feel) than the solution containing the (1S)-p-menthane-3,8-diol isomer mixture alone.

As is apparent from Examples 1 to 4 and Test Experiments 1 and 2, all the evaluations of the (1S)-p-menthane-3,8-diol isomer mixture alone, the (1RS)-p-menthane-3,8-diol isomer mixture alone, the combination of the (1S)-p-menthane-3,8-diol isomer mixture with menthol, and the combination of the (1S)-p-menthane-3,8-diol isomer mixture with vanillyl butyl ether were extremely good.

### Example 5

### Oral Cavity Composition (Toothpaste)

A toothpaste was prepared according to the following formula.

<Formula of Toothpaste>

| (Component) | (Blended amount: g) |
|---|---|
| 1-Menthol | 0.25 |
| (1S)-p-Menthane-3,8-diol isomer mixture | 0.05 |
| Calcium hydrogen phosphate (dihydrate) | 50.00 |
| Glycerin | 25.00 |
| Sodium lauryl sulfate | 1.40 |
| Carboxymethyl cellulose sodium | 1.50 |
| Sodium saccharin | 0.20 |
| Sodium benzoate | 0.10 |
| Strawberry-type flavor (manufactured by | 0.70 |
| Takasago International Corporation) | |
| Purified water | the balance |
| | Total 100.00 |

The toothpaste prepared according to the formula had a cooling and refreshing feel and no bitterness in the oral cavity. The toothpaste of the above-described formula had a longer-lasting cool and refreshing flavor than a toothpaste not using the (1S)-p-menthane-3,8-diol isomer mixture of the present invention, and the odor of the toothpaste of the above-described formula was persistent.

### Example 6

### Oral Cavity Composition (Tooth Gel)

A tooth gel was prepared according to the following formula.

<Formula of Tooth Gel>

| (Component) | (Blended amount: g) |
|---|---|
| 1-Menthol | 0.30 |
| (1S)-p-Menthane-3,8-diol isomer | 0.01 |
| mixture | |
| Sodium hydrogen carbonate | 97.69 |
| Magnesium oxide | 0.50 |
| Polyethylene glycol | 0.50 |
| Sodium saccharin | 0.20 |
| Trisodium phosphate | 0.10 |
| Peppermint-type flavor (manufactured by | 0.70 |
| Takasago International Corporation) | |
| | Total 100.00 |

The tooth gel prepared according to the above-described formula had a cooling and refreshing feel and no bitterness in the oral cavity. The tooth gel of the above-described formula had a longer-lasting, cool, and refreshing flavor with an impression of a prolonged flavor impact than a tooth gel not using the (1S)-p-menthane-3,8-diol isomer mixture of the present invention,

### Example 7

### Food and/or Beverage (Chewing Gum)

A chewing gum was prepared according to the following formula.

<Formula of Chewing Gum>

| (Component) | (Blended amount: g) |
|---|---|
| 1-Menthol | 0.01 |
| (1S)-p-Menthane-3,8-diol isomer mixture | 0.01 |
| Gum base | 24.00 |
| Corn syrup (42DE) | 6.70 |
| Glycerin | 1.10 |
| Sugar | 67.18 |
| Peppermint-type flavor (manufactured by | 1.00 |
| Takasago International Corporation) | |
| | Total 100.00 |

The chewing gum prepared according to the above-described formula had a cooling and refreshing feel and no bitterness. The chewing gum of the above-described formula had a longer-lasting, cool, and refreshing flavor with an impression of a prolonged flavor impact than a chewing gum not using the (1S)-p-menthane-3,8-diol isomer mixture of the present invention.

### Example 8

### Flavor and/or Fragrance Composition

A flavor and/or fragrance composition comprising the (1S)-p-menthane-3,8-diol isomer mixture was prepared in a usual manner according the following formula (the blended amounts are expressed in parts by mass).

<Formula of Flavor and/or Fragrance Composition>

| (Component) | (Blended amount: g) |
|---|---|
| Apple base (manufactured by Takasago | 8.0 |
| International Corporation) | |
| Bergamot oil | 14.0 |
| Ethyl acetoacetate | 5.0 |
| Methyl dihydrojasmonate | 23.0 |
| Laurinal | 3.0 |
| Levosandol (manufactured by Takasago | 4.0 |
| International Corporation) | |
| Orange oil | 8.0 |
| 10-Oxa-16-hexadecanolide | 8.0 |
| Phenoxanol (manufactured by IFF) | 6.0 |
| Styralyl acetate | 3.0 |
| Thesaron (manufactured by Takasago | 8.0 |
| International Corporation) | |
| (1S)-p-Menthane-3,8-diol isomer mixture | 30.0 |

### Example 9

### Cosmetic (Shampoo)

According to the following formula, 100 g of a shampoo scented with the above-described flavor and/or fragrance composition of Example 8 at 1.0% was prepared. This shampoo gave a cooling sensation, and the cooling sensation effect was persistent.

<Formula of Shampoo>

| (Component) | (Blended amount: g) |
|---|---|
| Polyoxyethylene lauryl ether sodium | 14.00 |
| sulfate | |
| Lauramidopropyl betaine | 4.00 |
| Coconut oil fatty acid diethanolamide | 3.00 |
| Cationized cellulose | 0.50 |
| Ethylene glycol distearate | 1.00 |
| Ethyl paraoxybenzoate | 0.25 |
| Citric acid | sufficient quantity |
| Flavor and/or fragrance composition of | 1.00 |
| Example 8 | |
| Purified water | the balance |
| | Total 100.00 |

### Example 10

### Sensory Stimulant Composition

A sensory stimulant composition comprising the (1S)-p-menthane-3,8-diol isomer mixture was prepared in a usual manner according to the following formula (blended amounts are expressed in parts by mass).

<Formula of Sensory Stimulant Composition>

| (Component) | (Blended amount: g) |
|---|---|
| (1S)-p-Menthane-3,8-diol isomer mixture | 50.00 |
| 2-(Menthoxy)ethanol | 20.00 |
| Menthyl 3-hydroxybutanoate | 15.00 |
| 3-Menthoxypropane-1,2-diol | 10.00 |
| Isopulegol | 4.89 |
| Spilanthol | 0.10 |
| Vanillyl ethyl ether | 0.01 |
| | Total 100.00 |

### Example 11

### Food and/or Beverage (Chewing Gum)

A chewing gum was prepared according to the following formula.

<Formula of Chewing Gum>

| (Component) | (Blended amount: g) |
|---|---|
| 1-Menthol | 0.01 |
| Sensory stimulant composition of Example | 0.01 |
| 10 | |
| Gum base | 24.00 |
| Corn syrup (42DE) | 6.70 |
| Glycerin | 1.10 |
| Sugar | 67.18 |
| Peppermint-type flavor (manufactured by | 1.00 |
| Takasago International Corporation) | |
| | Total 100.00 |

The chewing gum prepared according to the above-described formula had a cooling and refreshing feel and no bitterness. The chewing gum of the above-described formula had a more emphasized refreshing feel and a more persistent cooling feel than a chewing gum not using the sensory stimulant composition comprising the (1S)-p-menthane-3,8-diol isomer mixture of the present invention.

### INDUSTRIAL APPLICABILITY

The (1S)-p-menthane-3,8-diol isomer mixture or the (HIS)-p-menthane-3,8-diol isomer mixture used in the present invention serves as a cooling sensation component better in persistence of refreshing sensation or cooling sensation than a (1R)-p-menthane-3,8-diol isomer mixture, while retaining such a characteristic of not causing unfavorable stimulation, bitterness, or the like. Thus, the present invention provides a cooling sensation composition or a sensory stimulant composition comprising the compound and a flavor and/or fragrance composition, food, beverage, cosmetic, daily use product, oral cavity composition, or pharmaceutical comprising the cooling sensation composition or the sensory stimulant composition, which have no bitterness and are excellent in cooling sensation effect and sensory stimulation effect and also in persistence of these effects.

1. A cooling sensation composition comprising a p-menthane-3,8-diol isomer mixture 50% by mass or more of which is constituted of (1S)-isomers.

2. The cooling sensation composition according to claim 1, wherein
the mass ratio of the (1S,3R,4S) isomer:the (1R,3S,4R) isomer in the p-menthane-3,8-diol isomer mixture is 50:50 to 99.5:0.5.

3. The cooling sensation composition according to claim 1, wherein
90% by mass or more of the contained p-menthane-3,8-diol isomer mixture is constituted of the (1S,3R,4S) isomer and the (1S,3S,4S) isomer.

4. The cooling sensation composition according to claim 1, further comprising at least one cooling sensation component selected from the group consisting of menthol, isopulegol, menthone, camphor, pulegol, cineol, mint oil, 3-menthoxypropane-1,2-diol, N-alkyl-p-menthane-3-carboxamide, 3-menthoxy-2-methylpropane-1,2-diol, 2-(menthoxy)ethanol, 2-methyl-3-(1-menthoxy)propane-1,2-diol, 3-menthoxypropan-1-ol, 4-1-menthoxybutan-1-ol, menthyl 3-hydroxybutanoate, 1-(2-hydroxy-4-methylcyclohexyl)-ethanone, menthyl lactate, menthol glycerin ketal, N-methyl-2,2-isopropylmethyl-3-methylbutanamide, menthyl glyoxylate, menthyl succinate, menthyl glutarate, peppermint oil, spearmint oil, eucalyptus oil, mint oil, peppermint, and spearmint.

5. A sensory stimulant composition comprising:
the cooling sensation composition according to claim 1; and
at least one warming sensation component selected from the group consisting of capsaicin, gingerol, vanillyl butyl ether, vanillyl ethyl ether, vanillyl propyl ether, 4-(1-menthoxymethyl)-2-phenyl-1,3-dioxolane, 4-(1-menthoxymethyl)-2-(3′,4′-dihydroxyphenyl)-1,3-dioxolane, 4-(1-menthoxymethyl)-2-(2′-hydroxy-3′-methoxyphenyl)-1,3-dioxolane, 4-(1-menthoxymethyl)-2-(4′-methoxyphenyl)-1,3-dioxolane, 4-(1-menthoxymethyl)-2-(3′,4′-methylenedioxyphenyl)-1,3-dioxolane, 4-(1-methoxymethyl)-2-(3′-methoxy-4′-hydroxyphenyl)-1,3-dioxolane, vanillin acetals, capsicum pepper oil, capsicum pepper oleoresin, ginger oleoresin, nonylic acid vanillyl amide, jambu oleoresin, Japanese pepper extract, sanshool-I, sanshool-II, sanshoamide, black pepper extract, chavicine, piperine, and spilanthol.

6. A flavor and/or fragrance composition, food, beverage, cosmetic, daily use product, oral cavity composition, or pharmaceutical, comprising
the cooling sensation composition according to claim 1.

7. A p-menthane-3,8-diol isomer mixture 50% by mass or more of which is constituted of (1S)-isomers.

8. The p-menthane-3,8-diol isomer mixture according to claim 7, wherein
the mass ratio of the (1S,3R,4S) isomer:the (1R,3S,4R) isomer is 50:50 to 99.5:0.5.

9. The p-menthane-3,8-diol isomer mixture according to claim 7, wherein
the (1S,3R,4S) isomer and the (1S,3S,4S) isomer constitute 90% by mass or more of the entire composition.

10. A flavor and/or fragrance composition, food, beverage, cosmetic, daily use product, oral cavity composition, or pharmaceutical, comprising the p-menthane-3,8-diol isomer mixture according to claim 7 in an amount of 0.0001 to 90% by mass.

11. A flavor and/or fragrance composition, food, beverage, cosmetic, daily use product, oral cavity composition, or pharmaceutical, comprising
the sensory stimulant composition according to claim 5.
